(19) United States
(12) Patent Application Publication
(10) Pub. No.: US 2025/0019720 A1
(43) Pub. Date: Jan. 16, 2025

(54) Metamorphic Biomachine For Immortality

(71) Applicant: Tory Cortland Weston, Pendleton, IN (US)

(72) Inventor: Tory Cortland Weston, Pendleton, IN (US)

(73) Assignee: West Nanorobotics LLC, Pendleton, IN (US)

(21) Appl. No.: 18/350,618

(22) Filed: Jul. 11, 2023

(51) Int. Cl. C12N 15/86; C12N 15/70

(52) U.S. Cl. CPC C12N 15/86; C12N 15/70; C12N 2740/10043

(57) ABSTRACT

Metamorphic Bio-machines are a vector that inserts genes into bacteria and human cells and are also a type of plasmid that holds are stores DNA of up to 90,000 base pairs for constructed DNA repair pathways genes.

This is a Metamorphic Bio-machine and Metamorphic Plasmid that inserts 90,000 base pairs into animal and bacterial cells. These include Nerve Cells, White Blood Cells, and E. coli Bacteria.

### BRIEF SUMMARY

The Metamorphic Bio-machine and Metamorphic Plasmid is a construct of the hybrid between a bacteriophage and retrovirus vector. This construct uses the caspid of the bacteriophage to transfer the DNA into bacteria and the retrovirus vector's caspid modified with chickenpox caspid genes to increase the caspid size of the retrovirus vector to 90,000 base pairs. There are two sets of Pol genes have been modified with constructed protein sequences that are synthetic to be stored inside the bacteriophage caspid and make the transfer possible of both 90,000 base pairs and 170,000 base pairs by using Pol integration genes. The Rabies G-Protein is used for entry into the cell of 90,000 base pairs into human brain cells and retroviral glycoproteins which allow entry into white blood cells on the retroviral construct modified with several genes from different organisms to increase efficiency. The Storage plasmid for the DNA repair pathways of any animal are constructed by oligonucleotide synthesis as constructed DNA within the plasmid with surrounding retroviral LTR3′ and LTR5′ with all this used in conjunction will allow the transfer of 90,000 base pairs into human and bacteria cells specifically the genes that contain the synthetic sequence of DNA repair pathways.

### Reference Paragraph for Sequence Listing

XML Name: Sequence Listing XML
Date of Creation: Jul. 18, 2023
File Size: 38,000 bytes

### BRIEF DESCRIPTION OF THE FIGURES

FIG. 1 Sample Metamorphic Bio-Machine Gene Map, This is the circular gene map of a Metamorphic Bio-machine Vector.

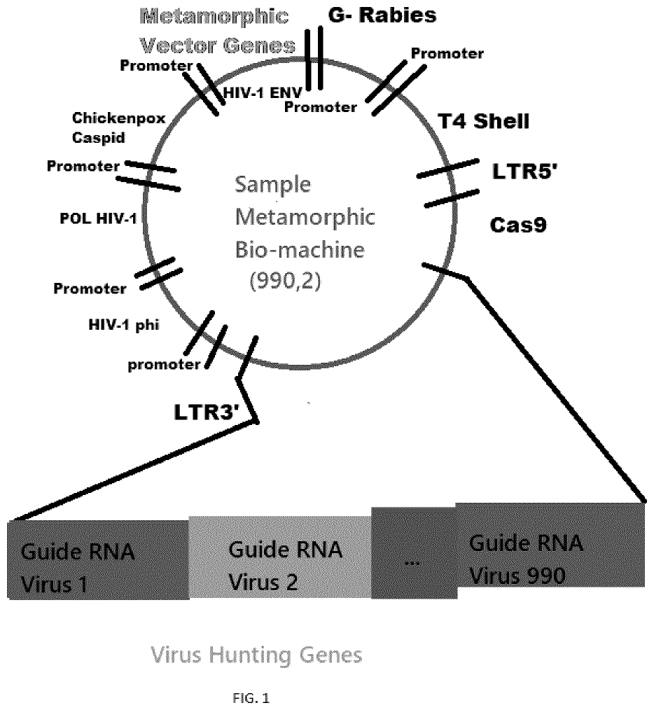

FIG. 2 Sample Metamorphic Plasmid Gene Map, This is a second circular gene map of a Metamorphic Plasmid.

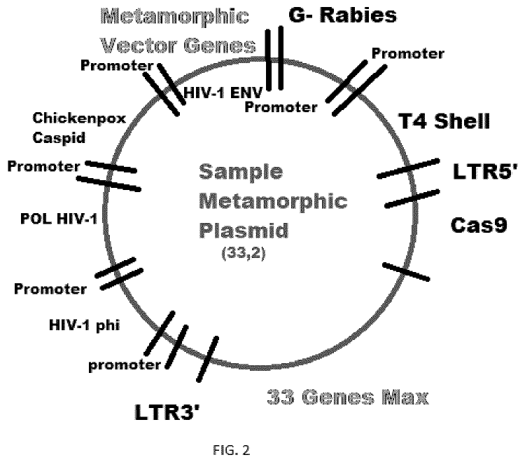

FIG. 3 Table with the Genes for DNA repair pathway, This is a table with DNA repair pathway genes.

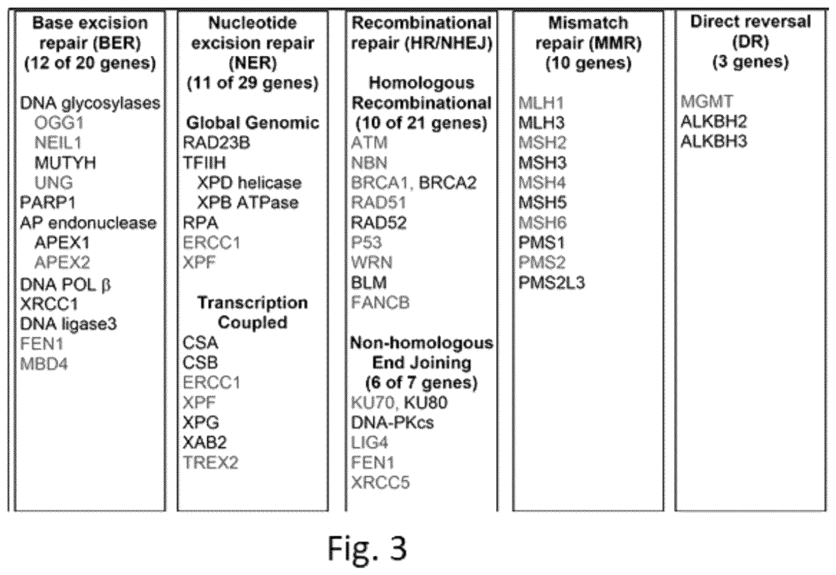

### DETAILED DESCRIPTION

There are several parts each part will be explained in detail they are HIV-Psi, Pol HIV-1, POL HIV-1 Modified, Chickenpox Caspid, HIV-1 ENV, G-Rabies, T4 bacteriophage shell, LTR3′, constructed DNA repair pathways, and LTR5′.

HIV-psi is the gene controlling the packaging of a retrovirus into the capsid. This is the gene that transfers the constructed DNA into the caspid it is a required component of the Metamorphic Bio-machine which allows integrase protein to uptake the construct's DNA from the LTR3′ and LTR5′.

Pol HIV-1 is a gene controlling the production of integrase and reverse transcriptase which moves the DNA into the cell of the retroviral construct part of the metamorphic bio-machine. This is the machinery that causes the RNA to be converted into DNA to be inserted into the nucleus of a human cell.

POL HIV-1 Modified is the HIV-1 POL gene modified to uptake DNA into the bacteriophage and transfer it into the bacteria via the lysogenic cycle which uses reverse transcriptase and integrase to transfer the genetic material into the bacteria cell. This is has been modified in such a way to allow the bacteriophage shell to uptake these proteins as they they were naturally apart of the bacteriophage's protein make up with modification with bacteriphage components to allow uptake into the bacteriophage shell.

Chickenpox Caspid is the caspid of chickenpox gene which can hold up to 90,000 base pairs of genetic material This has been genetically modified to resist external factors such as heat and cold being a extremophile version of the chickenpox capsid. This chickenpox capsid is made via controlled evolution of the chickenpox caspid by using heat and cold tempering of the chickenpox virus via a process.

HIV-1 ENV is the glycoproteins of HIV-1 being the GP genes of HIV-1. These genes allow entry of the Retroviral construct into the animal cells especially white blood cells. These are the targeting proteins that allow final entry into the cell for white blood cells.

Rabies—G Protein is the glycoprotein of Rabies that is the G Genes of Rabies. This is pseudotyped on the surface of the retroviral construct giving the construct a second set of glycoproteins that allow entry into nerve cells. This allows multi-valence being the DNA can be inserted into two different cell types.

T4 Bacteriophage Shell is the proteins essential for the formation of the T4 Bacteriophage structure. There are several genes that create the structure of the bacteriophage. This allows for transfer in a bacteriophage structure this is the outer shell of the bacteriophage component.

LTR5′ and LTR3′ are the two primers put around the DNA to be inserted to make the Pol genes and HIV-Psi take the DNA into the Metamorphic Bio-machine. These genes interact in such a way to bring the DNA into the cells when including the other processes but these must be placed around the genes that you want to insert such as the DNA repair pathways.

DNA Repair Pathways are the genes which there can be up to 32 genes.

### Transcription Coupled Repair

CSA, CSB, ERCC1, XPF, XPG, XAB2, and TREX2

### Mismatch Repair

MLH1, MLH3, MSH2, MSH4, MSH6, PMS1, PMS2, and PMS2L3

### Direct Reversal Repair

MGMT, ALKBH2, and ALKBH3

### Recombinational Repair

ATM, NBN, BRACA1, BRCA2, RAD51, RAD52, P53, WRN, BLM, and FANCB

### Non-Homologous End Joining

KU70, KU80, LIG4, FEN1, and XRCC5.

These are the genes for Transciption coupled Repair, Mismatch Repair, Direct reversal, Recombinational Repair, and Non-homologous end joining. These are DNA repair processes that repair various damaging effects such as replication errors, Free Radicals, UV Light.

This all forms the Metamorphic Bio-Machine that insert genes for DNA repair into bacterial and animal cells when the various genes are all constructed via oligonucleotide synthesis.

### Genetic Code in Constructed DNA

### How to Use

The genetic sequence of the Metamorphic Bio-machine must be inserted into anything with ribosomes and it will begin to produce the genes for the bio-machine then make the bio-machine that inserts the DNA repair pathway genes into the cell. The bio-machine must be purified from the producer cell line then put into a syringe and injected into the person or a culture of the person's cells that are being modified by the vector.

1. Any Metamorphic Bio-Machine which are vectors with the usage of a single vector or multiple vectors.

2. Any Metamorphic Plasmid which is a plasmid with multiple transfer vectors to insert DNA into host cells of two or more different types especially human and bacterial cells.

3. Any plasmid containing the genes for DNA repair pathways of any animal in constructed DNA.

4. The Method of claims 1, 2, 3 being the Metamorphic Bio-Machine being used to transfer the DNA repair pathways of any animal integrating them into a bacteria or human cell.


SEQUENCE LISTING (1 sequence)

SEQ ID NO: 1 - DNA, 36609 bp, synthetic construct
  source 1..36609: mol_type = other DNA
caaaggagtttctagccagaacttccgctggggactttccaggggaggtgtggctggggcggagttggggagtggctaaccctcagatgctgcataaaagcagccgcttttcgcttgtactgggtctctcttggtagaccaggtcgagcccgggagctctctggctaacaagggaacccactgcttaagcctcactctcaactgcttktagtgctctgagccckamgacctggcctcaaagggctgacccagccatacgtagatcctggggtctttctaggactgtgcctccctgcttgacctatgctccaggaccctgccctcaacccttgtcagctccctaagtcttcctgtttcccagtctcagattgagtgggkgctctcattgctctctcgacgcaggactcggcttgctgaagcgcgcacggcaagaggcgaggggcggcgactggtgagtacgccaaaaattttgactagcggaggctagaaggagagagatgggtgcgagagcgtcaatattaagcgggggatggatcgggtagaatcagaagaacccatggatggattcgaatcgcccgtattctccgaaaatacatcttctaattccggatggtgttccgacgcattttccgattcgtacatcgcttataatccagcccttctgctaaaaaacgatttgttattttcagaattgttatttgcctcccacttaataaatgttccccgtgcaatagaaaacaacgtcacttatgaggcctcttcggcggtaggtgtggataatgaaatgacctcaagtaccactgaatttatagaagaaattggagacgttttggcgttagacagagcctgtttggtctgtagaacgcttgatttgtataaacgtaaatttggactgacaccggaatgggttgcggactacgccatgttatgtatgaaaagtctggcatccccgccctgtgcagttgtcacttttagcgctgcctttgaatttgtgtatcttatggatcgttactacctgtgccgttataacgttactttggttgggtcctttgccaggcgcacgctttccctgttagatatacaaagacatttttttttgcatgtatgttttcgtaccgatggagggttaccaggtatacgaccgccccccggtaaggaaatggccaacaaagtaagatattccaattactccttttttgtacaggcggtagttagggctgcattactatcgatcagcacgtctcgtttagacgaaaccgaaacgcgtaagtcattttactttaatcaggacggactgactggaggccctcaacctttagcggccgccttggctaattggaaagattgcgcgcggatggttgactgttcatcatcggaacatcgcacaagtgggatgattacctgcgcggaacgtgcattaaaagaggatatagagtttgaagatatattaatagacaaacttaaaaaatcgtcttacgtagaagcagcttggggttacgcagacttggctttattattactgagtggggttgctacttggaatgtagacgagcgtacaaattgtgctatagaaactcgcgttggatgtgttaaatcatactggcaggcgaaccggattgaaaactccagggacgttccaaaacaattttccaaatttacgagcgaggatgcctgtcccgaagtagcatttgggcctattttgttaactaccttaaaaaacgcaaagtgccgtggtcgcacgaataccgaatgcatgttatgttgtttattaaccatagggcactattggatcgctttgcggcagtttaaaagggatatattagcatactcagcaaataacacaagtttatttgactgtatcgaacctgtaatcaatgcatggagcctagataaccccattaaacttaaatttccatttaatgatgagggtcgattcataaccattgtaaaagcagcaggttccgaggccgtatataaacatttattttgcgatctcctatgcgctctctcggaattacagacaaaccctaaaattttatttgcccatcctacaaccgcggataaggaagtgttggagttatataaagcccaactggctgcacaaaacagatttgaaggtcgtgtatgtgctggcctgtggacattggcgtatgcatttaaagcctaccagatttttccacgcaaaccaaccgccaatgccgcattcatacgagatggaggacttatgcttcgacgacatgcaatatcgctggtctccctcgaacacaccctatcgaagtatgtctagtcattttaaactctttctaagctgcttcttgaatgaagctattaattgtgttttggtgtcagattcattatattcaaatcctctttgaagcatctcggccatcatttcctcttttcctaaacgagaaaattcctttgatttatctccaacaaagttagggtgaatattattttgggtgtaatcggattttaaataagtaagtaaattttctaaccattcaagataatcaacaccttgtccctttaagccagaacgattaaatttatgcttcatttgaccttctgcagcattgcatagattacaaagcaatccacgcacctttcctgcttttggtccatttaattcatggtcatggtcgaggtgattagcttgaacatcaggatttagttctcgttggcaaattaagcatttaccgttttgtgcatcataaaatttctgtttttcttctttgtataatttgccagtcaataacattggaaggtcttgatataaacaaacttttagatatttctgacctccccggaattgacggggaggaaatcaaagtgtatgaacctctgcaattagtagaagttaaaagcaatccacaaaaccgtactccagacttagaagatgattatggagtagttcgtcgaaatatgcattttcagcaacaaatgctaatggacgctgccaagatttttcttgagacagcaaagaatgctgattctcctcgtcacatggaagtatttgcaactcttatggggcaaatgactacgacgaacagagaaatactgaagcttcataaagatatgaaagacattacatctgagcaggttggcaccaaaggcgctgttcctacaggtcaaatgaatattcagaatgcgacagtattcatgggttcaccaacagaattaatggacgaaattggtgatgcttacgaggctcaagaagctcgtgagaaggtgataaatggaacaaccgattaatgtattaaatgatttccatccgttaaatgaagctggaaaaattttaataaaacacccaagcttagcggaaagaaaagatgaagatggaattcattggataaaatctcagtgggatggaaaatggtatcctgaaaaattcagtgattaccttcgtctacacaaaatagtaaaaattccaaacaactctgataagcctgaattatttcaaacttataaagataagaataataaaagatctcggtatatgggtcttcctaacttgaaacgagctaatattaaaacacaatggactcgtgaaatggttgaggaatggaaaaaatgccgagatgatattgtttattttgcagaaacatactgtgccattactcatattgactatggtgtcataaaggttcaattacgtgactatcagcgtgatatgctcaaaataatgtcatctaaacgtatgactgtttgtaatctatcgcgccagctcggtaaaaccaccgtagtagctattttccttgcacactttgtatgttttaacaaagataaagctgtaggtattcttgcacacaaaggctcaatgtctgcggaagttttagaccgtactaagcaagcaattgaactgcttcctgactttttacaaccaggaattgttgaatggaataagggttcaattgaactagataatggttcttcaattggcgcttatgcttcctctcctgacgcagttcgtggtaactcgttcgcaatgatttacattgacgaatgtgcgtttattccaaacttccatgattcctggcttgctattcaaccagtaatttcatctggtcgtcgttcgaaaattattattactacgactcctaatggattaaatcatttttatgatatttggactgctgctgtcgaaggtaaatctggatttgaaccatatactgctatttggaattcagttaaagaacgtctttataacgatgaagatatttttgacgatggatggcaatggagcatacaaaccattaatggttcttcattagctcaattccgtcaagaacatactgcagcgtttgaagggacttctggtacattaatttcaggaatgaaattagctgttatggattttattgaagtaacaccagatgatcatggttttcaccaatttaaaaaacctgaaccagatagaaaatatattgcaactctagattgctcagaaggtcgtgggcaagattaccacgctttgcatattattgatgttactgatgatgtgtgggaacaggttggtgttttgcattcaaacactatttctcatttaattctacctgacatcgttatgcgttatttagtagaatacaatgaatgcccagtttatattgaattaaatagtactggtgtgtcagttgcaaaatcgctttatatggatttagaatacgaaggtgttatctgcgattcatatactgatttaggaatgaaacaaactaaacgcacgaaagcagtaggatgttccacgctaaaagaccttattgaaaaagataagcttattattcatcaccgagcgactattcaagaatttagaacgtttagtgaaaaaggcgtgtcttgggcggctgaagaaggttatcatgacgatttagtaatgtctttagtgatttttggatggttatcaacgcagtcaaaatttattgattatgcggataaagatgacatgcgattagcatctgaagtattttcaaaagagcttcaggatatgagcgacgactacgcgccagttatatttgtggattcggttcattctgctgagtatgttccagtatctcatggtatgtcaatggtataatttttagggaagatctggccttcccacaagggaaggccagggaatttccttcagagcagaccagagccatcagccccgccagaagagagcttcaggtttggggaagagacaacaactccctctcagaagcaggagccaatagacaaggaactgtatcctttagcttccctcagatcactctttggcaacgacccctcgtcgcaataaagataggggggcaactaaaggaagctctattagatacaggagcagatgatacagtattagaagaaatgagtttaccagggagatggaaaccaaaaatgatagggggaattggaggttttatcaaagtaagacagtatgatcaggtagccatagaaatctgtggacataaagctataggtacagtattaataggacctacacctgtcaacataattggaagaaatctgttgactcagattggctgcactttaaattttcccattagtcctattgaaactgtaccagtaaaattaaagccaggaatggatggtccaaaagttaaacaatggccattgacagaagaaaaaataaaggcattagtagaaatttgtacagaaatggaaaaggaaggaaaaatttcaaaaattgggcctgaaaatccatataatactccagtatttgctataaagaaaaaagacagtactaaatggagaaaattagtagatttcagagaacttaataagagaactcaagacttctgggaagttcaattaggaataccacatccagcagggttaaaaaagaaaaaatcagtaacagtactggatgtgggtgatgcatatttttcagttcccttagatgaggacttcagaaagtatactgcatttaccatacctagtacaaacaatgagacaccagggattagatatcagtacaatgtgcttccacaggggtggaaaggatcgccagcgatattccaaagtagcatgacaaaaatcttagagccttttagaaaacaaaatccagacatagttatctatcaatacatggatgatttatatgtaggatctgacttagaaataggacagcatagaataaaaatagaggaactgagacaacatctgttgaggtgggggtttaccacaccagacaaaaaacatcagaaagagcctccattcctttggatgggttatgaactccatcctgataaatggacagtacagcctatagtgctaccagaaaaagatagctggactgtcaatgacatacagaagttagtgggaaaactgaattgggcaagtcagatttatgcaggcattaaagtaaagcaattatgtaaactccttaggggaaccaaggcactaacagaagtagtaccactaacagaagaagcagagctagaactggcagaaaacagggaaattctaaaagaaccagtacatggagtgtattatgacccagcaaaagacttaatagcagagatacagaagcaggggcaaggccaatggacatatcaaatttatcaagagccatttaaaaatctaaaaacaggaaaatatgcaagaatgaggggtgcccacactaatgatgtaaaacaattaacagaggctgtgcaaaaagtagccatagaaagcatagtaatatggggaaagactcctaaatttaaactacccatacaaaaagaaacatgggaagcatggtggacagagtattggcaagccacttggattcctgagtgggagtttgtcaatacccctcccttagtgaagttatggtaccagttagagaaagaacccatagtaggagcagaaactttctatgtagatggggcagctaatagggaaactaaattaggaaaagcaggatatgttactgacagaggaagacaaaaggttgtccccctaacggacacaacaaatcagaagactgagttacaagcaattcatctagctttgcaggattcaggattagaagtaaacatagtaacagactcacaatatgcattaggaatcattcaagcacaaccagataaaagtgaatcagagttagtcagccaaataatcgagcagttaataaaaaaggaaaaggtctacctggcatgggtaccagcacacaaaggaattggaggaaatgaacaagtagataaattagtcagtgctggaatcaggaaagtactgtttctagatggaatagataaggcccaagaagagcatgaaaaatatcacagtaattggagagcaatggctggtgattttaacctgccacctgtggtagcaaaagaaatagtagcctgctgtgataaatgtcagctaaaaggagaagccatgcatggacaagtagactgtagtccaggaatatggcagctagattgtacacatttagaaggaaaagttatcctggtagcagttcatgtagccagtggatatatagaagcagaagttattccagcagagacagggcaggaaacagcatactttctcttaaaattagcaggaagatggccagtaaaaacaatacatacagacaatggcagcaatttcaccagtaatacagttaaggccgcctgttggtaggagatggcgttattcaatttgaggtacaacaacccatgatagcacgtgatggcccacatcccgtcgatcagcctgttcataattatatggttaagcggatacacaagcgttcgttaagcgctgcgtttgcaattgcatcggaagcgttgagtttgttaagtaacacatatgtcgatgggacagagattgactcatcgttacgtataagagctatccaacagatggctcgtaatttacgcaccgttttggactcatttgaacgaggcactgccgatcaacttcttggtgttctattggagaaagccccaccgctatcgctgctttcaccaattaataaattccaacccgagggacatctaaatcgtgttgcacgcgcggccctactttcggacctcaaacgtagagtctgtgcggatatgttttttatgacccgacacgccagggaacctaggctgatctctgcgtatctgtcggatatggtttcgtgcacccaaccatcggtgatggtatcacgaataactcatacaaacactcgcggacggcaggttgacggtgtgttggtaacaacagcaaccttaaaacggcaactattacagggaattttacaaattgacgacaccgccgctgacgtaccagtaacatatggcgaaatggttctacaggggacaaacttggtaaccgcccttgtgatgggaaaggccgtccgcggaatggatgatgtagcccgccatctccttgatataaccgaccctaacacgttaaacatcccgtctatacccccacaatccaactccgattcaacgacagctgggcttccggttaacgcccgtgttcctgcggatttagtgattgttggggataaacttgtattcttagaagcattagaacggcgggtctaccaagctacgcgcgttgcctaccctcttattggaaatatagatattacgtttatcatgccaatgggagtgtttcaggcaaactccatggacagatatacacgacacgccggcgatttttcaactgtatccgaacaggatccacgtcaatttccaccccaagggatttttttttataataaagatgggatattaacacagttgactcttcgtgatgcaatgggtaccatctgccacagttcattgcttgatgttgaggccacacttgttgccctccgccaacaacatttagatcgtcagtgttattttggtgtatacgtggccgagggtacagaggacacattggatgttcaaatggggaggtttatggaaacgtgggcagatatgatgcctcatcaccctcattgggtaaacgaacatttaacaattctacagtttatagctccgagcaacccgcgtctaaggtttgaattaaaccccgcctttgatttttttgttgcaccgggggacgtagaccttcccggaccgcagcgtcccccggaagccatgccaaccgttaacgcaacattacggattatcaacggaaacattcccgtgcctctatgtcccatttcatttcgagactgtcgcggaacccaactcggtttgggaagacatacaatgaccccggcaaccattaaagccgtaaaggatacatttgaagaccgcgcatacccaactattttctacatgctagaggctgttattcatggaaacgaaagaaacttctgtgcgttactgcgactgttaacacagtgtattcgcgggtattgggagcaatcccacagggtggcatttgtaaataactttcacatgttaatgtacataactacatatctcggaaacggtgagcttcccgaagtctgtattaatatatatcgggatttactgcagcatgtaagagcattacgccaaactataaccgattttacaatacaaggagagggccataacggcgagacctcggaagcgctaaataacatccttacggatgacacgtttattgcacctattctatgggattgtgatgcgttaatataccgtgatgaagccgcccgagaccgactccccgcaattcgtgtaagcgggcgaaacggataccaagcccttcactttgtggatatggccgggcataacttccaacgacgcgataatgtgttaatccacgggagacccgttcggggagacacgggtcagggtattcccattaccccacaccatgaccgtgaatggggtattctctccaagatttactactatattgtcattcctgcattttcccgcggttcctgttgtacaatgggcgtgcgttatgatcgcctataccctgcgttacaggcagttatcgttccggaaattcccgctgatgaagaagccccaactaccccagaagatccaagacaccctcttcacgcacaccaactcgttccgaactctcttaacgtttacttccataatgcacacctaaccgttgatggtgatgcattgctcacactacaagagttaatgggagatatggctgaacgaacgacggccattttagtatcaagcgcccccgatgcgggagccgccacggcaacaaccagaaatatgagaatatatgacggagcgctttaccatggccttattatgatggcatatcaggcgtacgatgaaaccattgcaacgggtacttttttttatcccgttccggtcaaccctctgtttgcatgtccggaacatttggcatcattgcgtggaatgacaaatgctaggcgggttttggcaaaaatggtaccaccaatccctccttttctgggagccaaccaccacgcaactatacgccaacccgttgcctaccatgtaacgcatagtaagtcggattttaatactcttacatattctcttcttggagggtattttaagtttacaccaatagctcttacacatcaactacgaacgggatttcaccccgggattgcctttaccgtagtgcgccaggatcgctttgccacagagcaacttttatatgccgagcgtgcttctgaatcgtactttgtcggacaaatccaagtacaccatcatgatgctattgggggggtaaactttaccctaacccaacccagagctcacgtggacctgggagtcgggtatacagctgtatgtgccacagcagccctgcgatgccctctcacggatatgggcaatactgcccaaaatctttttttttcacgaggaggagtgccaatgttacatgataacgttaccgaatcgttgcgtcgtataacagcatcggggggtcgcttaaatcccaccgaacccctacccatcttcggcggactacgtcctgctacatcggcaggaattgcacgagggcaagcctctgtgtgtgagtttgtggccatgccggtgtccactgacctacaatattttagaactgcatgcaatcctagaggtcgagcatctggaatgttatatatgggtgaccgtgacgttgatatagaggctataatgtttgatcacacacaatcggatgttgcttatacagatcgagcaactcttaacccatgggcatcacaaaaacattcatacggtgacaggctatacaacggaacatacaaccttacaggcgcttctcctatctacagcccatgctttaagttttttacaccagcggaggttaacactaattgtaatacactggatcggcttctaatggaggcaaaggctgtggcgtcgcaaagctccaccgacactgaatatcaatttaaacgccctcccggttctaccgaaatgacacaggatccgtgtggcctttttcaagaagcatatccaccactatgctcaagcgatgcggccatgttacgaacggctcacgcgggagaaaccggggcagatgaagttcacttagcccaatatctgattcgagacgcgtcgccccttaggggatgtcttcctcttccgcgataatttcaccacgcccacatacccactcccaataaaagccctgtagagcgcattggcatcttacttgagatttggatacgctcggccgacttggtctgtttcacgcttccttaaacaacatggctatgccatttgagatagaggtattgttaccaggagaactatccccggcggaaacatctgcattacagaaatgtgagggaaaaattattaccttctcaaccctgcgtcatcgagcttcactggtggatatagcgctgtcgtcatattacattaacggtgctccaccagacacgctctcgctgttagaggcataccgaatgcgattcgcggcagttataacacgggtcatcccgggaaagttgttggcgcatgccattggcgtgggtactcctacacccgggttgtttattcaaaatacatcccccgttgatctttgtaatggcgattacatctgcttacttcctccggttttcgggtccgcagactcaattcgcttggactctgtaggactggaaattgttttccctttaaccatcccccagaccttaatgcgagaaataatcgccaaagtggttgcacgggccgttgagcgcacggccgcgggtgctcaaattttaccccacgaagttctacgaggcgcggatgtcatttgttacaatggaaggcgttatgaactcgaaacaaatttacaacatcgggacggatcggatgcggctattcgcacattggttttaaatctaatgttttccataaacgagggatgtctgcttttattggcgctgattccaactttgttagtccaaggagcacacgacggttatgtaaatttattgatacaaacggccaattgcgttagagaaaccggccagttaattaatataccgccaatgccgcggattcaagacggccatcgccgatttcccatatatgaaactatttcatcttggatatcaacatcatctagactgggggataccttgggaactcgcgcaattttacgcgtctgtgtgtttgatggaccctctactgttcatccgggagaccgcacagccgtgattcaagtgtaaacaggtgttaataaaaacacaaccagtctagttacatttcacgcgtcttgtttttatttaataggcataaacacggaatccggtatacatgaactgccaatatacacggacataattaatgcaaccatcagatcatctgacattgttcccgtggtacctttacccgtgtaagtttttgtgtctagattacccataccgcctttaattacctctgtcaggttatccaactgtttacatagatactccacggggtctacacctaactttactgttagggatacaagctcctgtgaggctattatatttccggagttaaatcgtttaacaaaatagtctacggccggcgttttttgtttttgtaataaaaaaaaagggtacgccacgctacatccgggaggtatggaatgataaaacagtaacactggagcggaagatagcacgtttcccttttcgaggacagcaaactgttgtgctatagccaacgatatggcaactgcagaatcctggctgctgtttccctctatagaaacgtgtacgtttgtaaatgtattggggtgtaaagcgagtatgtggcctaagcattgagtaacgcaacgccctatctcactggaagacgtgccagttaaagctctaagaaaaaagtgctccaatccaaatataatccaatccgacttataacgaccaacaatcgctacaccagtaccagacgctcgtgtatttgaggtaaatgcagggtctacgtaaacgtacaacactgacgataatatagcacaattcgcaacggttgacggccggtataaaataaacctctcacgggcagtttttgtaaataatggccggtcaaaccccacacccccagaattctgtttacgcccacctacaatttcctgcacgaaggagtcggccataaataaatctgcagtgcgccgcatggctccatccattgtgatgaaaaccggcttatttaatacataacacgaacaagctgtgacatcgcttatttccagaaaagcaccgcccctaatacaactaccagtacaattacaacgaacagggcatatgtcacgttagctacgggtagagcaagtttccagacacgcgtagtttgggtatcgggtaacgcaggtttaatgtcactttgcatttgaacagacgtgtttggacttccgttctcgggtggggatctgaatgaaggccgccagcgtatatattcatccaaattattgccagtttccttatacatgtatgcatccgtggcgcgggccataagtttaatggtgcgagatggatcttccggtcccataaaacgaaaggataactgaacatatggcattcgcacaaagcagttcacccacattaaagcctggagaggtcggcggtcaatacccccacctcgtttaattgattccaaagcagataggttgataccggtacttaacgttgaactaagaatcacgttattactgtcaatggacacttcagccactggtgcgttagtcggacgaaaaaaaaaaccttgaaatagcacagacacccccgtattttgaatttttatgtaagggtcacaatctacttgcgcccaattcgccattaaacgcataatatactctaccggaaaggcttcggatacgttgtcttcgccgttaaactgaaaaacacaacgggcgggggggcgttgtggatcaaatattggaagatccccatcgcaacattgaagagcgcttggtaccaccaaccgaatacgttgtaaaagattatctccgcaacccctcctgcgttcactccgtacatacgttctccgtgacaatgagagtgaaggggatcaggaagaattatcagcactggtggagatggggcatcatgctccttgggatgtggatgatctgtagtgctgcagaaaaattgtgggtcacagtctattatggggtacctgtgtggaaagaagcaaccaccaccctattctgtgcatcagacgctaaagcatatgacacagaggtacataatgtttgggccacacatgcctgtgtacccatagaccccaacccacaagaagtatttttggaaaatgtgacagaaaattttaacatgtggaaaaataacatggtagaacagatgcatgaggatataatcagtttatgggatcaaagcctaaagccatgtgtaaaactaactccactctgtgttactttaaagtgcactgatttgaatgatactaataccaataatagtagtacttcggaaaataatactaatcccactattagtggtggggaagggatgggggaaggagagatgaaaaactgctctttcaatgttaccacaaacataagagataaggtgcagaaagaatatgctcttttctataaacttgatataataccaatagataatacaagctatgcattgagacattgtaacacctcagtcattacacaggcctgtccaaaggtatcctttgaaccaattcccatacattattgtgccccggctggttttgcgattctacaatgtaatgataagaagttcaatggaacaggaccatgttcaaatgtcagcacagtacaatgtacacatggaattaggccagtagtatcaactcaactgctgttaaatggcagtctagcagaagaagagatagtacttagatctgaaaatttcacaaacaatgctaaaaccataatagtacagctgaatgcatctgtagaaattaattgtacaagactcaacaacaatacaaggaaaagtataaggataggaccaggaagcacattttatgcaacaggagccataataggagatataagacaagcacattgtaacattagtagagaaaaatggaatgacactttaaaacagctagttataaaattaggagagcaatttgggaatagtaacataatagtctttaaacaatcctcaggaggggacccagagattgtaatgcacagttttatttgtggaggggagtttttctactgtaatacaacacaactgtctaatagtacttggcagaggtctgatggtacttggaatagaactggagggttaaatgaaactaaagaaaatatcacactcccatgcagaataaaacaaattataaacaggtggcaggaagtaggaaaagcaatgtatgcccctcccatcagtggactaattagatgttcatcaaatattacagggctgctattaacaagagatggggggaatgagaacaatgggaccaatgggactgagaccttcagacctgagggaggaaatatgaaggacaattggagaagtaaattatataaatataaagtagtaagaattgagccattagggatagcacccaccagggcaaggagaagagtggtgcagagagaaaaaagagcagtaacattcggagctctgttccttgggttcttgggagcagcaggaagcactatgggctgcacgtcaatgacgctgacggtacaggccagactattattgtctggtatagtgcaacagcagaataatttgctgagagctattgaggcgcaacagcatctgttgcaactcacagtctggggcatcaagcagctacaggcaagagtcctggctgtggaaaggtacctaagagatcaacagctcctggggatttggggatgctctggaaagctcatctgcaccactaatgtgccttggaatactagttggagtaataaatctgagaatgagatctgggataacatgacctggatggagtgggacagagaaattaacaattacacaaacttaatatacgacctacttgaaaaatcgcagaaccaacaagaaaaaaatgaacaggaactattggaattggataaatgggcaagtttgtgaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaaaaatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagcggacaaattagatgttcatcaaatattacagggctgctattaacaagagatggtggtaataacaacaatgggtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctgtagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttatagagctattcgccacatacctagaagaataggacagggcttggaaaggattttgctataaaaaaaaaaaaaaaaaaaaaaaaaaaaaaaaaaactattaacatccctcaaaagacttagggaaagatggttccgcaagctcttctgcttgtacccattctgggtttttcctcgtgtttcgggaaattccctatttacacgataccagacacacttggtccctggagcccgatcgatatacatcatctcagttgcccaaacaatttggttgtagaggacgaaggatgcaccaacctgtcagggttctcctacatggaacttaaagttggacacatctcagccataaaggtgaacgggttcacttgcacaggcgttgtaacagaggcagaaacctacactaactttgttggttatgtcaccaccactttcaaaagaaagcatttccgcccaacaccagatgcttgtagagctgcgtacaactggaagatggccggtgaccccagatatgaagagtctctacacagtccgtaccctgactaccattggcttcgaactgtaaaaaccacaaaggagtccctcgttatcatatctccaagtgtggtagatttggacccatatgacaactcccttcactcgagggtcttccctagcggaaagtgctcaggaataacagtatcttctgtctactgctcaactaaccacgattacaccgtttggatgcctgaaagtctgagactagggacatcttgtgacatttttaccaatagtagagggaagagagtatccaaggggagcaagacctgtggctttgtagatgaaagaggcctatataagtctctaaaaggcgcatgcaaactcaagttgtgtggagttcgtggacttagacttatggacggaacatgggtcgcgatgcagacatcaaatgagaccaaatggtgtcctcccgatcagttggttaatctgcacgaccttcgctcagatgaaatcgagcatcttgttatagaggagttggtcaagaaaagagaggagtgtctggatgcattagagtccatcataaccaccaagtcagtgagtttcagacgtctcagttatttaagaaaacttgtccccgggttcggaaaagcatataccatattcaacaagaccttgatggaggctgaagctcactacaagtcagtcaggacttggaatgagatcatcccctcaaaaggatgtttgagagttggagggaggtgtcatcctcatgtaaacggggtgtttttcaatggtataatattagggcctgacggtcatgttttaatcccagagatgcaatcatccctcctccagcaacatatagagttattggaatcctcagttattcccctgatgcacccccttgcagacccgttcacagttttcaaggacggcgatgagactgaggattttatagaagttcaccttcccgatgtgcacgaacaagtctcaggggttgacctgggtctcccgaactggggggagtatgtattactaagtgcagggaccttgattgccttgatgttgataattttcctaatgacatgttgtagaaaagtcgatcggccagaatctacacaacgcagtctcagagggacaggaaggaatgtgtcagtcacctcccaaagcgggaaattcataccttcatgggagtcgtataaaagtgggggtgagactggactgtgaagatttgtcatcttttcgacgcttcaagttctgaagataaccttccctctaagctggggggaatctctgagttcaatagtcctccttgaactccattcaacagggtggattcaaaagtcatgagactttcattaatcatctcagttgatcaaacaaggtcatgtagattctcataattcgggaaatcttctagtagtttcagtgaccgacagtgctttcattccccaggaactgatgtcaaaggttgttgacgggtcaagaggtatttctgatgactccgtgcgtgggcccggacagaggtcatagtacgtcccatgatagcggactcagcatgagtcgattgagaaaagtaatctgcctcccatgaaggacaccggcaataactcacaatcatcttgcatctcagcgaagtgtgcataattataaaggggctagatcatctaagcttttcagttgagaaaaaaaaaaaaaaaaaaaaaaaaacgaactgtttgaatccatgcttggtggattaaaagagctctttgttgaacacaacgttgttgttccagaagaatcagttgatgttgtagctgaaatggaagaagagctgcaagaacataaagaagaatcgcctcgtctgttcgaagaactgaatatgcgcgacgcatatatcaattatgtgcagcgtgaagtggcattgagcgaaagtactaaagatctgactgagtctcaaaaagaaaaagtctctgctctggtcgaaggtatggattattcagatgcattctcaagtaaattgagtgcaatcgtagaaatggtgaagaaatctaataaagatgaaagcactattactgagagtataaatactcctgatactgaagcagccggactgaatttcgtcactgaagctgtagaagataaagctgcacagggtgcagaagatattgtaagtgtatatgcgaaagtcgcatctcgtttctaattttaaaggttaacacaaatgactatcaaaactaaagctgaacttttgaacaaatggaagccattactggaaggtgaaggtttaccggaaattgctaatagcaaacaagcgattatcgctaaaatctttgaaaaccaggaaaaagatttccagacagctccggaatataaagacgaaaaaattgctcaggcattcggttctttcttaacagaagctgaaatcggtggtgaccacggttacaatgctaccaacatcgctgcaggtcagacttctggcgcagtaactcagattggcccagctgttatgggtatggtacgtcgtgctattcctaacctgattgctttcgatatttgtggtgttcagccgatgaacagcccgactggccaggtattcgcactgcgcgcagtatatggtaaagacccagtggctgccggtgctaaagaagcattccacccaatgtatggtccagatgcaatgttctctggtcagggtgctgctaagaaattcccagctctggctgctagcacacaaaccacagtaggtgatatctatactcacttcttccaggaaactggtactgtatatctgcaagcttctgttcaagtaacaatcgatgctggtgcgactgatgctgctaaattagatgctgaaattaagaaacaaatggaagctggtgcactggtagaaatcgctgaaggtatggctacttctatcgctgaactccaggaaggtttcaatggttctaccgataacccatggaatgaaatgggcttccgtatcgataagcaagttatcgaagctaaatctcgtcagctgaaagctgcttactctattgaattagcacaagacctccgcgctgttcacggtatggatgctgatgctgaactgtctggtattctggctacagaaattatgctggaaatcaaccgtgaagttgttgattggattaactactcagctcaggttggtaaatctggtatgaccctgactccgggttctaaagctggtgtatttgacttccaggacccaattgatattcgtggtgctcgctgggcgggtgaatcctttaaagctctgttgttccagattgacaaagaagcagttgaaattgctcgtcagaccggtcgtggtgaaggtaacttcattatcgcttcccgtaacgtagttaacgttttggcttcagttgataccggcatttcttatgctgcacagggtctggctaccggctttagcactgatactaccaagtcagtatttgctggtgttctgggtggtaaataccgcgtatatatcgaccagtatgctaaacaggattatttcactgtaggttataaaggtccgaacgaaatggatgctggtatttactatgctccatatgtagctctgactccgctgcgtggttccgatccgaagaacttccaaccggtaatgggattcaaaactcgttacggtatcggtatcaacccatttgcagaatccgctgctcaggctccggcttctcgcatccagagcggtatgccttctattctgaatagccttggtaaaaacgcttactttagacgtgtatatgttaaaggtatctaatctctaacgatagaaacacaattttagggaaccttcgggttcccttttttctattttatacgatagcaatcaggcatatcatccgcatttatccaattgcgaatagttttaggactaactttaaaatgctccgctgcgtaatcaggattatcaaatttaacgccctttatacatattggaataaattttttaataccaccaagtttttcagaaatagctttacgatgtgaaatcgatataggtttgttttttcgtggatgaacatgtgttttataatattcattgcgccctttaactcgcttcgcaatagtttcatcagattgcttaacgcctgtttttgcctttgatatttttcgtttagcttccacagtcattccttcttttgttcgtattgataacatattacgatatgaaggatcttgcaaatgaactataactggatttcctctgccaccaatagcagcattataggtatcagttctcataacgaattcctcattaactagtaaagcttccattttatacatctcctcagatgaggagaaagaataaagaatttcttttttaaagttatgaataccatattttttgatggattttttgatgtttacgccagaacccatataaccatcgttttcgtcaagagtagcatgagctccgatgtaaatttttccattgatgatattagtaatttgatatattaaatatttcattttaaacatcactccgtttgtatatgattataatatcatattactttggtcttgtaaataactttataaatagtattatatttcaacaaggaaaatacaatggctaaaatcaacgaacttctgcgcgaatcaaccacaacgaatagcaactcaatcggtcgcccaaatctcgttgctttgactcgcgctaccactaaattaatatattctgacattgtagcaacgcaaagaactaatcaacctgttgctgctttttatggtatcaaataccttaacccagacaacgaatttacatttaaaactggtgctacttacgctggcgaagctggatatgtagaccgagaacaaatcacagaattaacagaagagtctaaattaactctcaataaaggcgatttattcaaatataataatatcgtttataaagtattagaagatactccatttgctactatcgaagaaagtgatttagaattagctcttcagattgcaatcgttcttttaaaggttcgtctattttctgacgcagcgtcaacaagcaaatttgaaagctctgatagtgaaattgcggatgctagattccagattaataaatggcaaactgcagttaaatctcgtaaacttaaaactggcatcacagttgaattagcgcaagatttagaagcaaatggattcgatgctcctaatttcttggaagatttgcttgcaactgaaatggcagatgaaatcaataaagacattctgcagtctttgattacagtgtcaaaacgctataaagttacaggaattactgatagtggattcatcgatttgagttatgcatctgctcctgaagctggtcgttcattataccgaatggtatgtgaaatggtttcgcatatccaaaaagaatcaacttatacagcaacgttctgtgttgcttcagctcgtgccgctgcgattcttgctgcatcaggctggttaaaacataaaccagaagatgacaaatatctttcacaaaatgcctacgggttcttagctaatggtttaccgctttattgcgatactaacagcccattagattatgtaatcgttggcgtagtagaaaatatcggtgaaaaagaaattgttggatcaattttctatgctccgtatacagaaggtctcgacttagatgaccctgaacatgtaggtgcatttaaagttgttgttgatccagaaagcttacaaccatctatcggtttattagttagatatgctttatcagcaaatccttatactgtagcaaaagatgaaaaagaagcaagaataattgacggtggagacatggataaaatggcaggtcgttcagatttgtctgttttattaggtgttaagctaccaaaaattatcattgatgaataaaacaaagggacctttcggtccctttttatttaacttaccaactcaatccaagctggacgaagtacatcttgtaccattttaactaattcctttttaatcaaagaaggattatctgcttgagttagagtaataccttcacgagaagtttcttccaaaatatcttgaacagttagccccatcacctttccaaaatcctttggaccaatttcgccaattttagaaataacgttatttacgcggttcagtgtaacgtaacaagctaaaattcccaccaatttgttatcagcttctgatagctcaactttagctttaataggcttatcagactttttcttttcactaaatttagagttcttgcatttaatcgctacacgatttccattacgaagccaagaaggataacaaggtttcaatacatatccttcagcagtaaatacttcgccttttgcttcggcattccaaacgcatttatttgcatcaactaatccagcatggtctactgtaaaattataatcttggacgacagaatctaaatcatttggcaatttaataagctcttcaaatttaccgcgacctaaaagtggagccattttaaatttaaatgtattacagaatgattccatcatataatcatctacataagtcacatcaccgctttctgtagtaacaataatgtcaaatacataaaaatctttatcacaataatcaacattcttctgaatgccaggtccagcgaattcgccaaagacttgataagatacaaccgctgaggtttccataatatcttgtacagctttaatggaatcagcataattcttcaaaataatttcatacccaaagaaatcttcagcaggaagaatcggtccagtgcgtttagcgcaagtcactttatcacgctcaataatcaatgagaaatttgtgccgtgaatcttttcacgagctacccactccccaccagtcaatcccaagctataaagtttttcaataaatttagagttgtaatgattttcaagactgctatactttttaaacataattaatcctcaaaatgtaatttctaaccaatcaccatcacgctgatcactattgactttaaagctgaatccttcttttctcagccaatcaccaatttcttctgtaatcaatttatcacgagcaatacaataataattaaaatgtgttttaccttgttcagctgctttattagcaagttctgaaaaatctttaataaaacactctagcttaaactgtttactttttaatgctttttcgcgtaattgattagcaaaagattcattttcataaagatcatactgttccatttttcacctttttattgatatgtctttttctatagacaactttttctcgagcccataatacagccacttcttttgcctgtaagtttaattcacgagcaatttcaatgaatgactttccagactcatgaagagtaaacaccacaacctcagttctcataatcaatctcatgttatcgagttggtgccattatatacatcattttctgattgtgttttgtgtgctttcaaaatgaagaaaggggccgaagccccttatgattatggataggtatagatgataccagtttctaaagcagttttatgaatgatgtatccattacgcgattcttggacatcaacttctggatagtctttcatcatcttctggagagtgtaacgatgcaggtaatatttactatctgggtcgtcagttttccaatctttaccttcttcggtcattttttggatttcatccataacccaccaaccgcaccagatgtaagctgagctacggtgtggaagaggatgaacataaggtaattcaccttctggttctggagcaactttagccgtgactgttacattaccagttttggtaacggttacagggttataatctgcagcagtaacagttgcagtaacttcaatagtttgacttccaacagatgaggtatcgacagtatatacgttagttgacccttctacaggagaagaatctttcttccatgagtaagtaatttgtgcttcttctggagcacccgtaacattagccgtaaatgtagccgaagcatcttgctgaacattaatagaaggaggagtcaatgtaacctgtggattcattgtcttcttattaaccgttaatgatacttcattagaagtaacgcttagtgcatcataatctgtcgcggttacttgggctacgcatttaattctttttactccacttgtagttggagtatagctaaatgtagagttagtttctccaccaacttgtgaatcatctacataccactgatacgtagcagatgctccatcaggttgagaagctaaggcagcagtaaattgaactggggttccaatcactccagccgcaggactagcaggagttacggctaaggtagtcgtctgtgtcttatttttaactgtgatagttgttgtcgcttcagccgtttccgggcctccttcagaaagtgtatttgttgcaactactttaatagtcttttgaccggcaggtccttttagtacataactaaaagttgcttcagctccatcttgtggaacattatctacgctccaagcatatgtaatagttccgcctccagtttgaccactgggtgtagcagtaaactgcttagtttcatcaataacccctgtaggtgttttaggagttatatcaactgtaaaagtcataagttatccttattttaatgttacgaaagaagagttgcgtgtttcacgaattaaaactgatccatcgcgattaatgtaataaattaagctaaataaagtttggtgtgctgacgcatgttcaaaactagttgggtgagatttccaatcaggagtttcagcaatccattgataaatccaccaaggaacagtacagaatcctagattttttccaatcagttgaagattcggactaaagttttccggaagagtaaatacagacggcttttcagattcaataatctcagctacagcctgttcgaatttttcttcaacaaaaggagtatcttcaatcaaaacatcggtattttcaggaattttatccgtttctactacttcaattttaatgtcagatttaatcggagaatcaatcagaagtgctgcttctggattgacttcttcatcgtcatattttaatccctctgcggcatcagcagcatcaattaagtctttaatagataacccatcagtctctggcataggttcactagcgagcttctggagggcttcttcaatatcaacaacgatattatcaaaagatttattctttttgacctttataccaaactgttcagcatattcagctaatttagctttagcttctttgttatcatcaagagccttcagctctgcaatataatctttatctatcataatatttcctcagtataaatatagatatatttattactcggaaaatagtatgtaccactttgtatatgaaacaacaaatctaataaatggtaaaaagtatataggaaagcactctactgatgacttgaatgatggttaccttggttccggtaaggcaattcagcaggctataaagaaatatggtgaaaacaatttctctagaacaatactaaaagagtttaaaacttccgaagaagcgtacatgtatgaagaagaaattataactcctgaactaataaaaagcaaaaattattataatatgaaacctggtggaattggtggaattgttatgactacagatgttatagcaaagatgaaagaatcttccgctaaaagatttgaaaactcaccgggcacggtattaggtaaaacttgttatactaatggaactaaaaatatttttattaaacctggagaacttgttccagaaggatttgtaaaagggatggttcatcctaatagaaagtccagaaaaggatgtaaagtcaaaccgactaccacaggaactttttgggtcaataatggcgcaataaataaattaatacaaccagacggtattattcccgacggatttattaaaggtcgtctcatgaaaagagattctaaaggcaaatttagtaaggcataattagatatttgaccagactttgtttgcagagaaacgttttccttttgaaacgaactgctgaagtggcatcaacacaacgttcgcccagtctttcggggcgatttcaacaaggctacccataatattaccaggtatatatgccttaatcatttggtctgcacccctaaatcctttcacttgactccaatcaatttttaatttcgttttattagtaatagtaggtgtatttgcatattgctttaaaagctcttctaggaattgctgacgagctttaggtggaatatagtgcaagtttaatccgtacattaaattatgcttacctaaaccaaggtaaattatcaaaggaaatttatcccagtaaggaagagtttccttgtgtttagcatcataagcaaaagcatatattcgtcccggctgcgggcgaacaactttatgtccttttacttgcttaatagtttcagcaaaccactttctggttttattattaattgctgcgccttcattacgaattttatcacgcaatgtttgtctgaatgaatttatcataagcagttgtctttcttgcttattgagtttattcattggttttgattcaagtttttgaatcttttcagccgttttaattcctgaagcatattttgacattgctgaagtaaacgtagagtatttgattcctctttcttcagcaaattgctttcctgtcattccttttgctttggcctttttatattcaagacctatctgaatccatttcttttcgtttaatgattgcttaacctttggaacttggggagtgctttcattaattatttgaaaaatagccattgaaatttaatgtattaagtttgtttgctccatgggctaaaatggacgaacgaaattttaaagaccaagaaaaagaagatcttgtttccattacagccccaaagcttgatgatggagcaagagaatttgaagtaagctcgaatgaagctgcttctccttataatgctgcattccaaacaatttttggttcatatgaaccaggaatgaaaactactcgtgagcttattgatacatatcgtaatctcatgaataactatgaagtagataatgcagtttcagaaatcgtttcagatgctatcgtctatgaagatgatactgaagtcgtagcgttaaatttggataaatctaaatttagcccaaaaattaaaaatatgatgttagatgaatttagtgatgtattaaatcatctatcgtttcaacgaaaaggttctgatcattttagacgttggtatgttgattcaagaattttctttcataaaatcattgatccaaaacgtccaaaagaaggcataaaagaattacgtagattagaccctcgccaagttcagtatgttcgtgaaattataacagaaactgaagctggcacaaaaatagttaaaggttacaaagaatattttatatatgatactgcccatgagtcatatgcatgtgatggtagaatgtatgaagctggcacaaaaataaaaattcctaaagctgccgtcgtttatgcccattctggattagtcgattgttgcggtaaaaatatcatcgggtatttgcatcgtgctgttaaacctgctaaccaattaaaattattagaagatgctgtagtcatttatcgcattactcgtgctcctgaccgtcgtgtttggtatgtagacacaggtaatatgcctgctcgtaaagctgctgagcacatgcaacatgttatgaacacgatgaaaaaccgtgtagtatatgatgcatcaacaggtaaaataaaaaatcaacagcataatatgtctatgaccgaagactattggttgcagcgccgtgatggtaaagctgtgacagaagttgatactcttcctggtgctgataatactggcaatatggaagatattcgttggtttagacaagctctttatatggcattacgtgttcctctttcacgcattccgcaagaccaacaaggcggtgtgatgtttgattctggaactagcattacacgtgatgaattaacgtttgctaaatttattcgtgagttacagcacaagtttgaagaagttttcctagatccgcttaaaacaaatcttttgcttaaaggtataatcacagaagatgagtggaatgatgaaataaataatattaagatagaatttcatcgggatagctactttgctgagctcaaagaagcagaaattttggaacgaagaattaatatgctaaccatggcagaaccatttattggtaaatatatttctcacagaactgctatgaaagacattttgcagatgactgatgaagaaatagaacaagaagccaagcaaattgaagaagagtctaaagaggctcgtttccaagaccccgaccaagaacaagaggatttttattactccaagcaaagacaagacatacttgacttatgggtctataatacacaaggatacttccctgattggcaaaactacacaccagggccagggataagatacccattatgttttggatggtgctttaaactagtaccagtagatccaagagaagtagaggaggacaacaaagaagagaacaactgcctgttacaccccataagccagcatggaatggacgatgaagacaaagaagtgctgatgtggaagtttgacagtgccctagcacgaagacacatagcccgagagaagcacccagagtmctttaagractgctgacaaaggagtttctagccagaacttccgctggggactttccaggggaggtgtggctggggcggagttggggagtggctaaccctcagatgctgcataaaagcagccgcttttcgcttgtactgggtctctcttggtagaccaggtcgagcccgggagctctctggctaacaagggaacccactgcttaagccttttttcagaaagcataattggcaatgccagcaacattttttacagymamggtgcacgcaccagagagttgctactgtattatataatgatttaagtcccccgaacagggacttgaaagcgaaagttaatagggactcgaaagcgaaagttccagagaagttctctcgacgcaggactcggcttgctgagtgcacacagcaagaggcgagagcggcgactggtgagtacgccaattttttgactagcggaggctagaaggagagagatggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagattaaatagtgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggcgaaactaaatcgtgaagatttgctgcgcaagcaacggacctttgacaacggctctattccccatcaaattcacttgggtgagctgcatgctattctgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgtgagcgtatgaaacgtattgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttcattaaagacgattcaatagacaataaggtcttaacgcgttctgataaaaatcgtggtaaatcgaataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatcttaatgccgtcgttggaactgctttgattaagaaatatccaaaacttgaatcggagtttgtctatggtgattataaagtttatgatgttcgtaaaatgattgctaagtctgagcaggaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttagaaaagacttaatcgttaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccacttttatccatcaatccatcactggtctttatgaaacacgcattgatttgagtcagctaggaggtgactga